(19) United States
(12) Patent Application Publication
(10) Pub. No.: US 2021/0252518 A1
(43) Pub. Date: Aug. 19, 2021

(54) BIOLOGICAL SAMPLE HOLDER AND HANDLER

(71) Applicant: QCDx LLC, Farmington, CT (US)

(72) Inventors: Triantafyllos P. Tafas, Rocky Hill, CT (US); Seth Winfree, Plainfield, IN (US); Scott W. Reed, Winchester, CT (US)

(21) Appl. No.: 17/132,046

(22) Filed: Dec. 23, 2020

Related U.S. Application Data
(63) Continuation of application No. PCT/US19/39244, filed on Jun. 26, 2019.
(60) Provisional application No. 62/690,402, filed on Jun. 27, 2018.

(51) Int. Cl. B01L 9/06; G01N 33/487; G01N 21/64

(52) U.S. Cl. CPC B01L 9/065; G01N 33/487; B01L 2300/0838; B01L 2300/0654; G01N 21/6458

(57) ABSTRACT

The present invention provides a biological sample holder and handler system for cell-based liquid biopsies. The system is useful for performing diagnostic assays, based on a simple blood sample. For example, the system is useful for delivering precision cancer diagnoses that improve patient outcomes by optimizing treatment options, monitoring therapy efficacy, characterizing metastasis and assessing treatment toxicity

### CROSS-REFERENCE

The present application is a continuation of International Application No. PCT/US19/39244, filed Jun. 26, 2019, which claims the benefit of U.S. Provisional Application No. 62/690,402, filed Jun. 27, 2018, each of which is entirely incorporated herein by reference.

### FIELD OF THE INVENTION

The present invention provides a biological sample holder and handler system for cell-based liquid biopsies. The system is useful for performing diagnostic assays, such as those based on a simple blood sample. For example, the system is useful for delivering precision cancer diagnoses that improve patient outcomes by optimizing treatment options, monitoring therapy efficacy, characterizing metastasis and assessing treatment toxicity.

### BACKGROUND OF THE INVENTION

Traditional solid tissue biopsies are the gold standard for cancer diagnosis. However, these assays are static and require fixation and staining of the samples for histologic analysis by conventional microscopic techniques. This limits sample size and thickness and does not present the opportunity to more readily sample and monitor tumors under dynamic conditions. For ongoing monitoring of disease progression and patient management, liquid biopsies offer a unique advantage because they involve non-invasive, blood-based testing of cell-free DNA (cfDNA) or circulating tumor cells (CTCs). Molecular analysis of cfDNA (including circulating tumor DNA, ctDNA) has shown high sensitivity and specificity for detecting driver mutations, structural rearrangements, copy number aberrations and changes in DNA methylation, therefore providing valuable information for disease monitoring.

However, a driver gene does not kill a patient. Rather, aggressive tumor cells do. CTC analysis permits the study of whole cells, and offers DNA, RNA and protein-based molecular profiling, as well as the opportunity for functional studies that can guide precision therapies.

CTCs have been detected in the blood of cancer patients in frequencies of 1:10⁸ to 1:10⁶ or higher. CTCs are released in the circulation from the primary tumor following a series of biological events also involving epithelial to mesenchymal transition (EMT). Single tumor cells or tumor cell clusters leave the primary tumor site, invade the blood vessels, and travel throughout the body until they leave the blood stream. The cells settle in different tissues, thereby generating the bud of metastasis formation. Detection of CTCs in the blood is challenging, both because of their scarcity and because they can express different phenotypes. These phenotypes include epithelial, mesenchymal and stemness-like CTCs, but with features that can change over time, converting from one state to another, and vice versa. See, Joosse et al (2015). Biology, detection, and clinical implications of circulating tumor cells. EMBO Mol Med 7: 1-11.

One of the early commercial CTC analysis systems was CellSearch® (Silicon Biosystems/Menarini) developed in the mid-2000's. This system is based on epithelial cell adhesion molecule (EpCAM) for CTC enrichment, followed by immunofluorescent staining for cytokeratin (positive CTC characterization), CD45 (which is also known as lymphocyte common antigen) to exclude leukocytes and diamidino-2-phenylindole (DAPI) for nuclear counterstaining. CellSearch validation studies showed the prognostic value of CTCs and led to FDA clearance for monitoring patients with metastatic breast, prostate and colon cancer. See, Hayes DF, et al. Circulating Tumor Cells at Each Follow-up Time Point during Therapy of Metastatic Breast Cancer Patients Predict Progression-Free and Overall Survival Clin Cancer Res 2006; 12(14): 4218-4224; de Bono, J S, et al. Circulating Tumor Cells Predict Survival Benefit from Treatment in Metastatic Castration-Resistant Prostate Cancer. Clin Cancer Res 2008;14:6302-6309; and Cohen S J, Punt C J A, Lannotti N, et al. J Clin Oncol. 2008;26(19):3213-3221.

Since the development of early commercial systems, multiple studies and trials with different endpoints have analyzed the clinical utility of CellSearch CTC enumeration. It was shown that increased CTC numbers (5 per 7.5 mL of whole blood) are associated with poor prognosis in metastatic breast cancer (MBC). A pooled study of trials conducted between 2003 and 2012 and involving 2000 MBC patients, confirmed the independent prognostic effect of CTC-count on progression-free survival (PFS) and overall survival (OS). It also confirmed that CTC-count improves the prognostication of MBC when added to full clinicopathological predictive models, which cannot be done with serum tumor markers. See, Bidard, F C (2014). Clinical validity of circulating tumor cells in patients with metastatic breast cancer: a pooled analysis of individual patient data. Lancet Oncol. 15:406-14.

Beyond enumeration, several studies addressed the question of genotypic and phenotypic characterization of CTCs. In metastatic breast cancer (MBC), because there are several drugs that target human epidermal growth factor receptor 2 (HER2) and are beneficial for patient management, testing for HER2 at the DNA, m.RNA and protein levels has been extensively used. In metastatic renal cancer, dynamic changes of live versus. apoptotic CTC subpopulations were shown to be a predictive marker of response to chemotherapy. See, Pestrin et al. (2012) Final results of a multicenter phase II clinical trial evaluating the activity of single-agent lapatinib in patients with HER2-negative metastatic breast cancer and HER2-positive circulating tumor cells. A proof-of-concept study. Breast Cancer Res Treat 134: 283-289. In early breast cancer, several studies including more than 2800 patients, have shown that the detection of CTCs is independently associated with poor prognosis. See, Franken et al (2012). Circulating tumor cells, disease recurrence and survival in newly diagnosed breast cancer Breast Cancer Res, 14:R133.

Liquid biopsies offer non-invasive, precision blood-based testing of circulating tumor DNA (ctDNA) or circulating tumor cells (CTCs). CTC liquid biopsies were shown to have prognostic value in different metastatic cancers [1,2,3], early disease [12] and to aid in management of metastatic cancer [10,13]. Current systems utilize CTC enrichment by antibody capture [4], filtration [5], dielectrophoresis [6] or other methods. Different CTC enrichment approaches impede direct comparison of results between studies while variable CTC phenotypes have different metastatic potential [7]. Quantitative detection and characterization of viable CTCs, may be valuable for the biology of cancer metastasis [8]. Single CTC isolation and whole genome analysis (WGA) is promising for targeted therapy selection and monitoring disease progression [9,11].

Almost every CTC diagnostic system utilized today requires enrichment to increase CTC abundance from less than or equal to about 1:10⁶ in the patient blood to about 1:1000 nucleated cells which makes microscopic examination feasible. Without an enrichment step, it is very difficult to otherwise find and qualitatively or quantitatively assess the CTCs. After enrichment, the specimen is stained for CTC-specific biomarkers by immunostaining or in situ hybridization. Enrichment methods either utilize a cell surface marker (such as EpCAM) for antibody-based CTC capture or some physical separation method such as filtration. Either approach is making selective assumptions that lead to incomplete detection of all CTCs. Since CTCs are known to contribute to metastasis formation, it is important to identify and characterize them as accurately as possible.

CTC detection, including live characterization and characterization without enrichment, and using biomarkers for multiple phenotypes, will open the way for a standardized CTC definition and benefit precision cancer diagnosis.

For the present invention we have developed an automated microscope that offers deep quantitation of all cells in a blood or other body fluid specimen using fluorescence Selective Plane Illumination Microscopy (SPIM) [14]. SPIM microscopy uses laser-sheet illumination which can offer resolution comparable to confocal microscopy with much less phototoxicity. After staining for multiple biomarkers, cells are mixed in a hydrogel solution (e.g. agarose), aspirated and allowed to immobilize in a transparent tube (e.g., a FEP tube). By means of a fixture, they are placed in the aqueous-solution filled chamber which is part of the SPIM optical path. Three-dimensional cell images are acquired in multiple fluorescent channels and every cell in the specimen, e.g. white blood cells (WBCs), are analyzed for the detection of target cells (e.g. CTCs). The system can observe cells while perfused with substances contained in the surrounding aqueous solution. In analytical validation experiments, the system is able to detect cancer cells at a frequency of <1:500.000 blood cells.

Therefore, it is seen that the state of the art is limited either by conventional solid tissue biopsy methods or by CTC analysis systems that require a cumbersome enrichment step which can add error to the methodology. The present invention, which utilizes a new application of selective plane illumination microscopy (SPIM) provides a rapid analysis system for characterizing and quantifying CTCs under more realistic biological conditions without the need for an enrichment step, and therefore provides a useful alternative to conventional solid tissue biopsy methods. The present invention is centered on a cell sample holder and handler system for use with SPIM.

### SUMMARY OF THE INVENTION

The present invention provides a biological sample holder and handler system for cell-based liquid biopsies. The system is useful for performing diagnostic assays, based on a simple blood sample. For example, the system is useful for delivering precision cancer diagnoses that to improve patient outcomes by optimizing treatment options, monitoring therapy efficacy, characterizing metastasis and assessing treatment toxicity.

The present invention relates to an apparatus for characterizing or quantitating particles in a biological sample comprising:

(a) a cylindrical chamber with a fluid input port and a fluid output port, wherein said chamber further comprises
(i) a first opening capable of allowing for illumination or excitation of the sample through an illumination lens (said illumination lens having a light path) mounted in the first opening, and
(ii) a second opening capable of allowing for observation of the sample, through a detection lens (said detection lens having a light path) mounted in the second opening,
   wherein the first opening and the second opening are oriented at 90 degrees (orthogonally) to each other to permit the orthogonal and coplanar orientation of the optical axes of the light paths of the illumination lens and the detection lens to each other,
   (b) a porous, light transparent capillary tube for containing the sample, said tube being open at both ends, such that the sample is capable of being in fluid connection with the cylindrical chamber,
   (c) an illumination or excitation source for the sample, and
   (d) an observation means for the sample,
   wherein the capillary tube is disposed within the cylindrical chamber such that the sample is capable of being oriented within the intersection of the light paths of the illumination lens and the detection lens.

In further embodiments the present invention relates to an apparatus wherein the capillary tube comprises a plurality of pores or holes.

In further embodiments the present invention relates an apparatus wherein the capillary tube has an inner bore diameter of from about 0.5 mm to about 10 mm.

In further embodiments the present invention relates to an apparatus wherein the capillary tube has an inner bore diameter of about 1 mm.

In further embodiments the present invention relates to an apparatus wherein the plurality of pores or holes of the capillary tube each have a diameter from about 0.002 mm to about 0.05 mm.

In further embodiments the present invention relates to an apparatus wherein the illumination or excitation source is a light sheet source.

In further embodiments the present invention relates to an apparatus wherein the light sheet source is a laser light sheet source.

In further embodiments the present invention relates to an apparatus wherein the observation means is a microscope.

In further embodiments the present invention relates to an apparatus wherein the microscope is a microscope for performing selective plane illumination microscopy (SPIIM).

In further embodiments the present invention relates to an apparatus wherein the observation means is a digital camera, a UV/visible spectrophotometer, or a raman spectrophotometer.

In further embodiments the present invention relates to an apparatus for characterizing or quantitating particles in and further manipulating and isolating particles from a biological sample comprising:

(a) a cylindrical chamber with a fluid input port and a fluid output port, wherein said chamber further comprises:
(i) a first opening capable of allowing for illumination or excitation of the sample through an illumination lens (said illumination lens having a light path) mounted in the first opening,
(ii) a second opening capable of allowing for observation of the sample, through a detection lens (said detection lens having a light path) mounted in the second opening, and
(iii) an access port for removing the particles from the sample.
   wherein the first opening and the second opening are oriented at 90 degrees (orthogonally) to each other to permit the orthogonal and coplanar orientation of the optical axes of the light paths of the illumination lens and the detection lens to each other,
   (b) a porous, light transparent capillary tube for containing the sample, said tube being open at both ends, such that the sample is capable of being in fluid connection with the cylindrical chamber,
   (c) an illumination or excitation source for the sample,
   (d) an observation means for the sample,
   (e) a means for positioning, moving, and extruding the sample from the capillary tube, and within the sample chamber, and
   (f) a means for removing the particles from the sample,
   wherein the capillary tube is disposed within the cylindrical chamber such that the sample is capable of being oriented within the intersection of the light paths of the illumination lens and the detection lens, and the sample is further accessible to the (f) means for removing particles from the sample.

In further embodiments the present invention relates to an apparatus wherein the (e) means for moving and positioning the sample is a syringe.

In further embodiments the present invention relates to an apparatus wherein the syringe of (e) is capable of being operated, moved, and rotated by a motorized device.

In further embodiments the present invention relates to an apparatus wherein the (f) means for removing the particles is a micropipette.

In further embodiments the present invention relates to an apparatus wherein the micropipette of (f) is capable of being operated, moved, and rotated by a motorized device.

In further embodiments the present invention relates to an apparatus wherein the biological sample is selected from bodily fluids such as for example, blood, urine, semen, saliva, amniotic fluid, spinal fluid, and seminal fluid.

In further embodiments the present invention relates to an apparatus wherein the biological sample is blood.

In further embodiments the present invention relates to an apparatus wherein the particles are selected from circulating tumor cells (CTCs), blood cells, rare immune cells (such as for the diagnosis of autoimmune disease), and pathogenic cells.

In further embodiments the present invention relates to an apparatus wherein the particles are circulating tumor cells (CTCs).

In further embodiments the present invention relates to an apparatus for characterizing or quantitating particles in a biological sample comprising the following components as illustrated in any of FIGS. 1 through 8:

glass syringe, glass capillary tube, a prepared sample in agarose gel, fluid output port, sample chamber and O-ring, illumination and detection lenses, lens holder, fluid manifold, micropipette, manipulator for the micropipette, and fluid connectors (input and output), wherein the capillary tube (for example a glass capillary tube) comprises one or more holes (for example micro laser drilled holes) to enable the introduction of reagents and washing steps to and from the capillary tube.

In further embodiments the present invention relates to a method for characterizing and quantitating target cells in a blood sample utilizing the apparatus of the present invention comprising the steps of:

(a) obtaining a blood sample from a subject,
(b) preparing the sample by one or more of the following steps (i) through (vi), comprising
(i) centrifugation to separate cell layers from the blood serum layer
(ii) removal of one of the cell layers,
(iii) suspension of the removed layer from step (b)(ii) in a buffer,
(iv) purification of the suspended layer of (b)(iii),
(v) immunostaining and/or fluorescence in situ hybridization (FISH) staining of the purified layer of (b)(iv), and
(vi) immobilization of the purified and immunostained and/or fluorescence in situ hybridized (FISH) layer of (b)(v),
   (c) subjecting the prepared sample from (b) to selective plane image microscopy by scanning the sample with a laser sheet light source at a multiple of cross sections to obtain contiguous cross-sectional images,
   (d) collecting or digitizing (e.g. with a digital camera) a sufficient quantity of contiguous cross-sectional images,
   (e) compiling the contiguous cross-sectional images to produce a composite image (such as a three-dimensional image), and
   (f) assessing the composite image to characterize and quantitate the blood sample for any target cells.

In further embodiments the present invention relates to a method comprising the further step (g) of collecting one or more target cells.

In further embodiments the present invention relates to a method for determining or diagnosing a disease state in a subject utilizing the apparatus of the present invention comprising steps of:

(a) obtaining a biological sample from a subject,

(b) subjecting the biological sample to selective plane illumination microscopy by scanning the sample with a laser light sheet source at a multiple of cross sections to obtain contiguous cross-sectional images,

(c) collecting a sufficient quantity of contiguous cross-sectional images,

(d) compiling the contiguous cross-sectional images to produce a composite image (such as a three-dimensional image), and

(e) assessing the composite image for the presence of selected target cells, and

(f) making a diagnosis based on the assessment from step (e).

In further embodiments the present invention relates to a method for determining or diagnosing a disease state comprising the further step (g) of collecting one or more target cells.

In further embodiments the present invention relates to a method for determining or diagnosing, and further treating a disease state in a subject utilizing the apparatus of the present invention comprising the steps of:

(a) obtaining a biological sample from a subject,

(b) subjecting the biological sample to selective plane illumination microscopy by scanning the sample with a laser light sheet source at a multiple of cross sections to obtain contiguous cross-sectional images,

(c) collecting a sufficient quantity of contiguous cross-sectional images,

(d) compiling the contiguous cross-sectional images to produce a composite image (such as a three-dimensional image), and

(e) assessing the composite image for the presence of selected target cells,

(f) making a diagnosis based on the assessment from step (e), and

(g) treating the subject based on the diagnosis from step (f).

In further embodiments the present invention relates to a method for determining or diagnosing, and further treating a disease state comprising the further step (h) of collecting one or more target cells.

In further embodiments the present invention relates to such methods wherein the subject is a human subject.

In further embodiments the present invention relates to such methods wherein the subject is an animal subject, including mammals such as mice, rats, dogs, and other mammals used in cancer research.

In further embodiments the present invention relates to a method wherein the disease state is cancer.

In further embodiments the present invention relates to a method wherein the selected target cells are circulating tumor cells (CTCs).

In further embodiments the present invention relates to a method wherein the CTCs are characterized.

In further embodiments the present invention relates to a method wherein the CTCs are quantitated.

In further embodiments the present invention relates to use of the apparatus of the present invention in the manufacture of a medicament for characterizing and quantitating selected target cells in a biological sample.

In further embodiments the present invention relates to a method which does not require enrichment or concentration of the sample for the target cells.

In further embodiments the present invention relates to a method wherein the sample comprises about 1 or less target cells per about 1×10⁶ total cells (i.e., total nucleated cells) in the sample.

In further embodiments the present invention relates to 1 method wherein the sample comprises about 1 or less target cells per about 1×10⁵ total cells (i.e, total nucleated cells) in the sample.

In further embodiments the present invention relates to a method wherein the sample comprises about 1 or less target cells per about 1×10⁴ total cells (i.e., total nucleated cells) in the sample.

In further embodiments the present invention relates to a method wherein the sample comprises about 1 or less target cells per about 1×10³ total cells (i.e., total nucleated cells) in the sample.

These and other embodiments of the present invention will become apparent from the disclosure herein.

### BRIEF DESCRIPTIONS OF THE DRAWINGS

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows a drawing for the sample holder and of the sample handler of the present invention.

FIG. 2 shows an exploded view of the components for the sample holder and sample handler of FIG. 1. Illustrated are: glass syringe, glass capillary tube, a prepared sample in agarose gel, fluid output port, sample chamber and O-ring, illumination and detection lenses, lens holder, fluid manifold, micropipette, manipulator for the micropipette, and fluid connector (input).

FIG. 3 shows a close up view of the sample tube of the sample holder of the present invention. Illustrated is a glass capillary tube with micro laser drilled holes to enable the introduction of reagents and washing steps. The illustrated tube has a diameter of about 0.7 mm with the laser drilled holes arranged over about 10 mm of the tube length.

FIG. 4 shows a cut away view of the system of the present invention and some of the motions available.

FIG. 5 shows a cut away view of the sample chamber with the sample ready for aspiration of cells from the sample.

FIG. 6 shows a view of the sample chamber.

FIG. 7 shows a cut away view of the sample chamber.

FIG. 8 shows an exploded view of the sample chamber, sample, capillary tube, and means for advancing the sample.

### DETAILED DESCRIPTION OF THE INVENTION

The biological sample holder and handler system of the present invention comprises several components and utilizes advanced characterization and quantitation techniques to provide high resolution, accuracy, and sensitivity.

### Selective Plane Illumination Microscopy (SPIM)

Fluorescence light sheet microscopy (FLSM) is a fluorescence microscopy technique in which a sample is illuminated by a laser light sheet (i.e. a laser beam which is focused in only one direction) perpendicularly (i.e. orthogonally or 90 degrees to the direction of observation. The light sheet can be created using e.g. cylindrical lens or by a circular beam scanned in one direction to create the light sheet. As has been reported, only the actually observed section of a sample is illuminated. Therefore, this method is reported to reduce the photodamage and stress induced on a living sample. Also, it has been reported that the good optical sectioning capability reduces the background signal and thus creates images with higher contrast, comparable to confocal microscopy. Furthermore, selective plane illumination microscopy (SPIM) and fluorescence microscopy techniques in which a focused sheet of light serves to illuminate the sample have become increasingly popular in developmental studies. Fluorescence light-sheet microscopy bridges the gap in image quality between epifluorescence microscopy and high-resolution imaging of fixed tissue sections. In addition, high depth penetration, low bleaching and high acquisition speeds make light-sheet microscopy ideally suited for extended time-lapse experiments. See, Huisken et al (2009). Selective plane illumination microscopy techniques in developmental biology. Development 136, 1963-1975 doi:10.1242/dev.022426. FLSM systems can be purchased from various companies such as Zeiss, Leica, or Olympus. These systems can also be built for research purposes following designs offered through open source groups such as OpenSPIM or SPIM-fluid.

See, http://openspim.org/Welcome_to_the_OpenSPIM_Wiki; and https://doi.org/10.1364/BOE.6.004447.

### Systems and Methods of the Present Invention

The system and method of the present invention can accurately detect epithelial, mesenchymal, or stemness-like CTCs, including intermediate phenotypes, because it is designed to quantitatively detect multiple CTC biomarkers. It provides fully automated CTC detection in patient blood samples for clinical diagnosis, academic research and drug development. The present system does not require enrichment because its high-resolution and high-speed microscope can scan and analyze every nucleated cell from the patient sample and deliver very sensitive detection of CTCs or other blood cell subpopulations such as T-cells.

The system has the unique ability to observe live cell preparations in addition to detecting and characterizing CTCs without enrichment. The system enables (i) spatial and temporal characterization of disease progression, and (ii) real-time observation of live CTC phenotypes by ex vivo imaging.

The system of the present invention can include an aqueous solution filled cell observation chamber. This enables observation of either fixed or live cell preparation. The chamber can be equipped with a media recirculation system which enables perfusion of the cells with solutions that can contain: (i) biomarkers such as antibodies or fluorescence in situ hybridization (FISH) probes appropriately labeled for enumeration and quantitation, (ii) substances for staining DNA or other molecules, (iii) agents including therapeutic substances, viral suspensions etc. that can affect the physiology of targeted live cells, (iv) de-staining solutions, and (v) cleaning and de-contamination solutions.

The system's “lossless” CTC detection is applicable to different cancer types. By scanning every nucleated cell from the blood sample and utilizing multiple markers associated with different CTC phenotypes. The system enables detection of epithelial, mesenchymal and stemness-like CTCs. Quantitative imaging of biomarker levels also allows detection of CTCs transitioning between different CTC phenotypes.

Unlike other microscopic methods, the system's high-resolution microscopy has very low phototoxicity (i.e. the light-induced degradation of photosensitive components or in general adverse light-induced effects), which permits multiple imaging sessions of a given specimen, in successive time points.

The system of the present invention includes a cell aspiration device that allows removal of target cells from the specimen (including while live), for further molecular, single-cell testing. CTCs isolated by the system can be used as a tissue source for drug sensitivity testing by utilizing subsequent ex vivo cultures and for the detection of specific mutations in CTC-derived cell lines. In CTC-derived cell lines, cells can be studied for their resistance to specific chemotherapy or targeted therapies or combinations of the above. Drug sensitivity testing can be carried out also in mouse xenograft models. The clinical utility of the CTC models can depend on (i) the percentage of patients in which CTC will be detected and (ii) whether the CTC models can reliably capture response to different drugs. The system and methods of the present invention can aid in combining CTC genomic and transcriptomic analyses together with drug sensitivity testing in CTC-derived cell lines and mouse models; this can provide new insights for driving personalized cancer treatment.

### Biological Sample Holder and Handler

The holder and handler of the present invention is a system that allows ex vivo observation of cells that have been stained with vital stains for CTC-specific biomarkers and maintained alive for periods of time supported by a 3-dimensional culture subsystem. A specially designed cell chamber will be fitted for input and output of culture media, gas regulation and control of environmental variables (temperature, pH etc). This will allow ex vivo observation of cells while perfused with culture media which may contain various substances. The chamber will be fitted with a micromanipulator (handler) used to isolate target cells under direct observation. Both the chamber and the micromanipulator can be operated automatically by a system computer and software system.

The ex vivo liquid biopsy will offer longitudinal observation of target cells, e.g. CTCs and WBCs and assessment of desired and undesired toxicity of therapeutic drug cocktails before used for patient treatment. This will drive precision medicine for improved outcomes and reduced adverse effects to the patient. Cell isolation will enable CTC genomic and transcriptomic analysis that may reveal improved therapeutic options, tuned to the patient's current disease status.

The sample holder and handler of the present invention, combined with deep quantitation of every cell the specimen, have the potential to become an important precision medicine tool. Deep CTC characterization and single-cell, genomic/transcriptomic analysis will enable the oncologist to select a treatment that is synchronized with the current disease stage. Ex vivo assessment of how a selected drug or drug combination affects CTCs and WBCs in the patient's blood will have to be studied against patient outcomes. However, it has the potential to revolutionize therapy selection and longitudinally, help in turning cancer from a devastating to a chronic disease.

A central computer system (not shown) operates a software package that (a) acquires and processes images of the biological specimen's features for identification and quantitation, (b) actuates the motorized components, pumps, sensors of the system, (c) operates a robotic arm that loads and unloads samples, and (d) handles digital information managed in local or wide area networks. The central computer system may utilize local or distributed processing protocols.

The system also includes or is coupled to a tunable laser source or multiple single wavelength laser sources, complete with light management optical path(s). An optical system modulating the light sheet can combine bilateral illumination to produce the sheet illumination for SPIM.

Imaging is done by illuminating the specimen with narrow spectrum excitation light provided by monochromatic and/or tunable laser sources. Images of the resulting emission are acquired by high sensitivity monochrome cameras on a field by field basis. These images are combined in 3-dimensional stacks, which are then analyzed for quantitative measurement of biomarker levels in the individual cells.

In operation, a biological specimen that can include live cells is stained with a variety of markers against proteins, nucleic acids or other cellular components and encased in an appropriately shaped cylindrical sheath to be fitted on a biological sample holder. The preparation is made by mixing the cell suspension with agarose or other hydrogels compatible with preserving the subcellular structure of the embedded cells, at a temperature where the solution is still liquid. In addition to the cells, fluorescent beads that serve the role of fiducial reference for the identified cells are added to the solution. The liquid cell/bead/gel suspension is aspirated in tubing that is chosen to be transparent to the fluorescence light regime utilized. After being allowed to solidify, the specimen can be visualized in the light path. The biological specimen is mounted on a specimen holder loaded onto the microscope stage.

All combinations of the foregoing concepts and additional concepts discussed in greater detail below (provided such concepts are not mutually inconsistent) are part of the inventive subject matter disclosed herein. In particular, all combinations of claimed subject matter appearing at the end of this disclosure are part of the inventive subject matter disclosed herein. The terminology used herein that also may appear in any disclosure incorporated by reference should be accorded a meaning most consistent with the particular concepts disclosed herein.

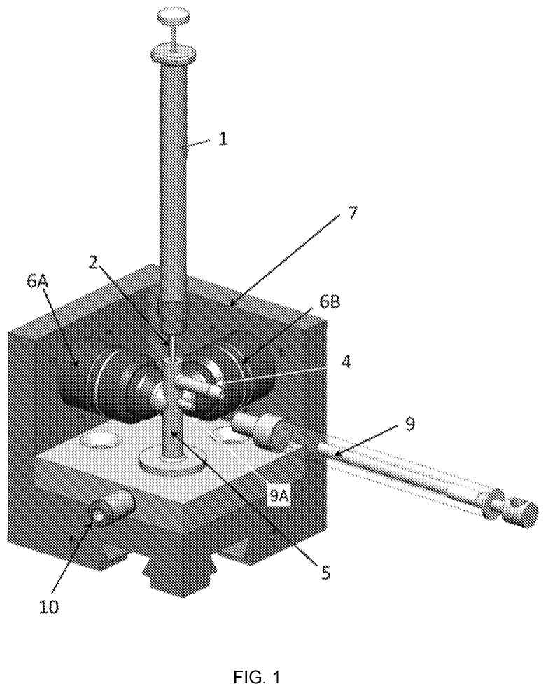

FIG. 1 shows a drawing for the sample holder and of the sample handler of the present invention. Shown is a means 1 for advancing and manipulating the sample 3 (not visible in this FIG. 1) contained within a sample holder such as a capillary tube 2 with a plurality of holes 2A (the capillary tube is not visible in this FIG. 1). The means 1 can be any of a variety of mechanical devices, including, for example a glass syringe. Shown is the cylindrical sample chamber 5, with a fluid output or outlet port 4, a lens holder 7, holding an illumination lens 6A and a detection lens 6B (which are oriented orthogonally, i.e. at 90 degrees to each other), and an access port 9A built into the cylindrical sample chamber 5 for allowing access for a device for retrieving particles of interest, such as a micropipette 9. A fluid input connector 10 is shown on the base of the lens holder 7. Not visible is the fluid input orifice, of the cylindrical sample chamber 5 located in the base of the chamber. In further embodiments, the means for advancing the sample can be controlled by an external motor, such as a 4-D motor 13 (not shown in this FIG. 1) to provide movement and control in the X, Y, and Z axes, as well as to provide for rotation of the sample. It is important that the optical axes of the lenses 6A and 6B are orthogonal and co-planar such that the sample chamber and sample can be positioned at the intersection of the respective optical axes for the lenses.

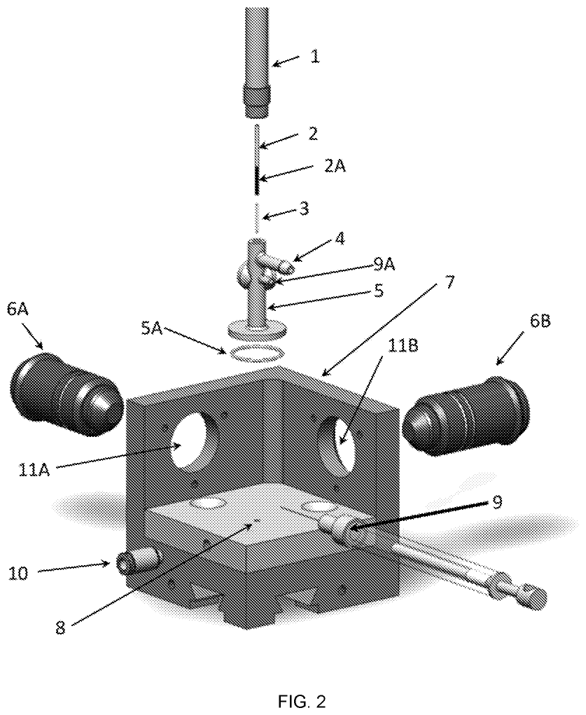

FIG. 2 shows an exploded view of the components for the sample holder and sample handler of FIG. 1. Illustrated are a means 1 for advancing the sample 3, i.e. a syringe such as a glass syringe 1, a capillary tube 2 where the capillary tube has a porous area, such that it is perforated with pores or holes 2A (this porous region is indicate but the individual holes are not visible in this FIG. 2). The sample 3 can be e.g. a biological sample prepared in an agarose gel. The cylindrical sample chamber 5 is shown with an optional O-ring 5A for providing a tight seal between the sample chamber 5, and the fluid manifold 8 of the base of the lens holder 7. The sample chamber has an output or outlet port 4. Also shown are an illumination lens 6A and a detection lens 6B and where they would fit in apertures 11A and 11B in the lens holder 7. Note the orthogonal orientation of the apertures for positioning the lenses 6A and 6B. The illumination lens 6A allows for illumination or excitation of the sample, wherein light from an illumination or excitation source (not shown) would be focused through the illumination lens 6A. The detection lens 6B allows for observation of the sample, wherein light emitted from the sample would be focused through the detection lens 6B to a detection means, such as a digital camera or a spectrophotometer (both not shown). Also, shown is a fluid input connector 10 on the lens holder 7, and a micropipette 9 and the access port 9A on the sample chamber 5 for the micropipette.

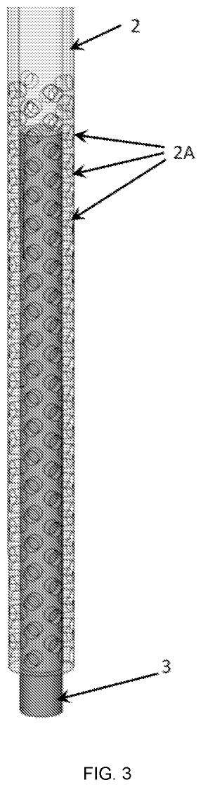

FIG. 3 shows a close up view of the sample tube 2 which is inserted into the cylindrical sample chamber 5 of the present invention. Illustrated is a porous capillary tube 2, open at both ends, with pores or holes 2A over at least a portion of the tube to enable the introduction of reagents and to permit washing steps for the sample. The pores or holes can be, for example, laser drilled holes. In some embodiments, the tube has an inner diameter or bore from about 0.5 mm to about 3 mm, with a convenient diameter being about 1 mm. The pores or holes through the wall of the tube are generally arranged over about 10 mm of the tube length. The holes can have a diameter from about 0.002 mm to about 0.5 mm. The capillary tube can have a length from about 10 mm to about 250 mm. Also shown in this FIG. 3. is a biological sample immobilized in a hydrogel, such as e.g., an agarose gel, 3. It should be noted in this FIG. 3 that the sample has been advanced past the bottom of the capillary tube where particles in the sample can be accessed with the micropipette 9 via the access port 9A. It is important that the micropipette can access the sample at the intersection of the optical axes of the light paths from the illumination and detection lenses.

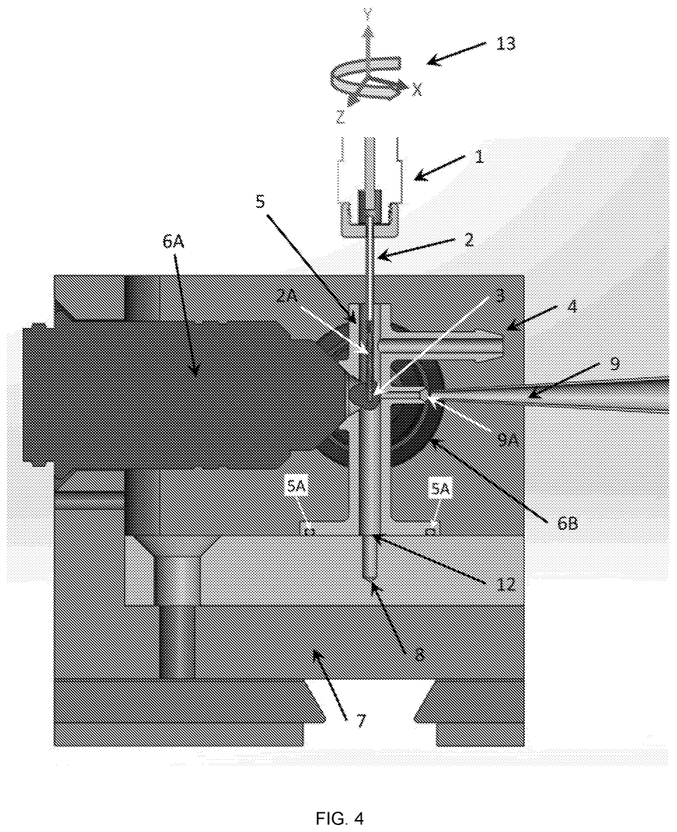

FIG. 4 shows a cut-away view of the system of the present invention and illustrates a means for moving and manipulating the sample with a syringe 1. The movement of the syringe 1 can be controlled by a 4-D motor, 13 (the actual motor is not shown but just illustrated where it can be placed), which can allow for motions in the X, Y, and Z axes, as well as for rotation. This motion is ultimately translated to the sample 3. The capillary sample tube 2 oriented within the sample chamber 5. The pores or holes 2A of the capillary sample tube are shown. The lens holder 7 is shown along with the illumination lens 6A and the detection lens 6B and their relative orthogonal (i.e. 90 degree) orientation to each other. Also, the fluid input area 12 at the bottom of the sample chamber 5 is shown, as well as a partial view of the fluid manifold 8. The optional O-ring 5A is indicated.

A micropipette 9 for extracting cells or particles of interest and an access port for the micropipette 9A on the fluid chamber 5 is shown. It is important that the micropipette can access the sample at the intersection of the optical axes of the light paths from the illumination and detection lenses.

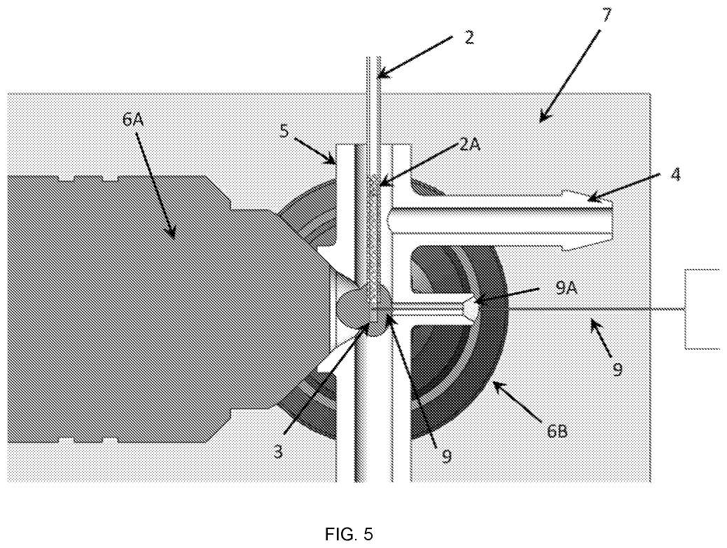

FIG. 5 shows a close up view of the sample chamber 5 with the sample 3 partially extruded from the bottom of the capillary tube 2 and ready for removal or aspiration of particles or cells from the sample via the micropipette 9 (note the needle portion of the micropipette) which is inserted into an access port 9A on the sample chamber 5. The pores or holes 2A on the capillary tube 2 are also indiated. The lens holder 7 is also indicated as well as the illumination lens 6A and the detection lens 6B. Also shown is the outlet port 4 of the sample chamber 5. It is important that the micropipette can access the sample at the intersection of the optical axes of the light paths from the illumination and detection lenses.

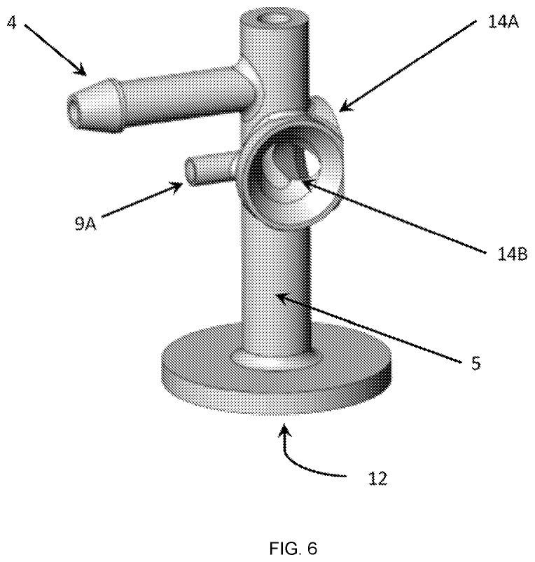

FIG. 6 shows a view of the sample chamber 5. The first 14A and second 14B openings are indicated. Note their orthogonal and co-axial orientation to permit the orthogonal and coaxial placement of the illumination lens 6A (not shown) and detection lens 6B (not shown) within the openings. The output or outlet port 4 and the access port 9A for the syringe 9 (not shown) are indicated. The position of the inlet port 12 is indicated in the base of the chamber, but is not visible in this view. The cylindrical sample chamber 5 can have a variety of dimensions. A nonlimiting range of dimensions is a height from about 5 cm to about 30 cm, an outer diameter from about 2 cm to about 4 cm, and an inner bore diameter from about 1 cm to about 3 cm.

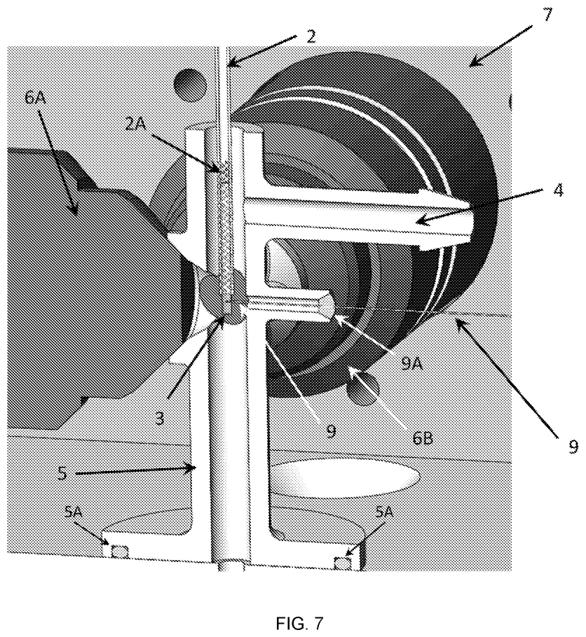

FIG. 7 shows another cut away view of the sample chamber 5 shown from an angle or perspective to show part of the sides of the illumination lens 6A and the detection lens 6B, which are mounted in the lens holder 7. The thin needle portion of the micropipette 9 is shown inserted into the access port 9A and into the sample 3. The capillary tube 2 is shown as well as the pores or holes 2A and the outlet port 4 of the chamber 5. Also indicated is the optional O-ring 5A.

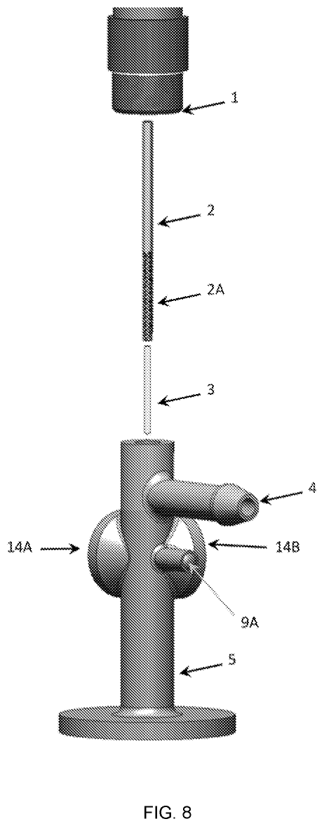

FIG. 8 shows an exploded view of the sample chamber 5, sample 3, capillary tube 2 and the portion of the tube with the pores or holes 2A, and means for advancing the sample 1, in this case a syringe. Also shown is the outlet port 4, the access port 9A for the micropipette 9 (not shown), and the first and second openings 14A and 14B, into which the illumination lens 6A and the detection lens 6B can be positioned.

In the foregoing embodiments, it should be noted that the illumination lens 9A and the detection lens 9B, as well as the illumination and detection means, can be swapped around, so long as their orientation is orthogonal. Also, the openings 11A and 11B of the lens holder and the openings 14A and 14B of the sample chamber would also be concurrently swapped in such a situation.

### Methods and Use of the Holder and Handler

A cell suspension can be observed in SPIM instrument mounted in fixture and embedded in hydrogels that allow cell perfusion with fluorescently labeled antibodies, fluorescence in situ hybridization FISH probes, and other stains as well as media that can sustain ex vivo cell observation.

### Selection of Embedding Gel and Specimen Fixture for SPIM Cell Suspensions

The following steps are performed: Compare performance of embedding gels including agarose, collagen, polyacrylamide and tubing such as micro-perforated, fluorinated polyethylene (FPE) and glass both for fixed and live cells. Optimize fixation/permeabilization protocols. Assess need of antifading for fluorescence bleaching. Adapt SPIM image acquisition to materials chosen. Quantitative analysis of cell staining and morphology changes via 3d image analysis with QCDx imaging software.

### Viability of Live Cell Preparations in Longitudinal Imaging Sessions.

Explore prototype chamber design fitted with computer-controlled, microfluidic media circulation, gas exchange mechanism and environmental sensors. Evaluate vital fluorescence stains for immunostaining and nuclear counterstaining and viability stains. Quantitative morphological changes in target cells to establish acceptable longitudinal ex vivo imaging periods. The holder and handler has design requirements for the chamber, specimen fixture, tube and embedding gel that will enable longitudinal imaging of cell suspensions, fixed or ex vivo.

### Application

The present invention can comprise instruments and kits for the detection and characterization of CTCs and other target cell populations

### References:

The following references have been cited above corresponding to the following numbering.

1. Hayes et al., Clin Cancer Res. 2006; 12(14): 4218-4224.

2. Cohen et al., J Clin Oncol. 2008; 26(19):3213-3221.

3. de Bono et al., Clin Cancer Res 2008; 14:6302-6309

4. Bidard et al., Lancet Oncol. 2014; 15:406-14.

5. Zhaomei et al., Int. J. Mol. Sci. 2016; 17,1665; doi:10.3390/ijms17101665

6. Peeters et al., Br. J. Cancer 2013; 108: 1358-1367.

7. Joosse et al., EMBO Mol Med 2015; 7: 1-11.

8. Alix-Panabieres et al., Nature Reviews Cancer 2014; 14: 623-631.

9. Ferrarini et al. PLOS ONE 2018; doi:10.1371/journal.pone.0193689

10. Scher et al., JAMA Oncol. 2016; doi:10.1001/jamaonco1.2016.1828

11. Dittamore et al., J Clin Oncol. 2018; 36, (suppl; abstr 5012).

12. Franken et al., Breast Cancer Research 2012; 14:R133.

13. Zhang et al. BMC Cancer 2016; 16:526; doi:10.1186/s12885-016-2578-5

14. Huisken et al. Development 2009; 136, 1963-1975; doi:10.1242/dev.022426

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity’ such as “either”’ “one of” “only one of” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### Equivalents

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

### Incorporation by Reference

The entire disclosure of each of the patent documents, including certificates of correction, patent application documents, scientific articles, governmental reports, web sites, and other references referred to herein is incorporated by reference herein in its entirety for all purposes. In case of a conflict in terminology, the present specification controls.

1-37. (canceled)

38. A device comprising:
(a) a chamber configured to receive a container, wherein the container holds a biological sample, and wherein the chamber comprises:
(i) a first end configured to receive at least a portion of the container;
(ii) a second end; and
(iii) an interior wall connecting the first end and the second end, wherein the interior wall comprises:
(1) a first portion disposed between the first end and the second end, wherein the first portion is configured to permit directing of a light towards the biological sample when the biological sample is disposed within the chamber; and
(2) a second portion disposed between the first end and the second end, wherein the second portion is configured to permit detection, by a detector, of the biological sample when the biological sample is exposed to the light;
(b) an optical source coupled to the chamber and positioned at the first portion of the interior wall, wherein the optical source is configured to direct the light through the first portion and towards the biological sample; and
(c) the detector, wherein the detector is coupled to the chamber and positioned at the second portion of the interior wall, and wherein the detector is configured to detect the biological sample through the second portion upon the exposure to the light.

39. The device of claim 38, wherein the first portion of the interior wall has a first optical axis, wherein the second portion of the interior wall has a second optical axis, and
wherein, if the biological sample is extruded out of the container while the biological sample is within the chamber, then the chamber holds the extruded biological sample at an intersection of the first optical axis of the first portion and the second optical axis of the second portion.

40. The device of claim 39, wherein the first optical axis of the first portion and the second optical axis of the second portion are substantially perpendicular to each other.

41. The device of claim 38, wherein the first portion of the interior wall and the second portion of the interior wall are disposed at a substantially same distance between the first end and the second end of the chamber.

42. The device of claim 41, wherein the chamber further comprises a third portion, wherein the third portion is disposed at the substantially same distance between the first end and the second end of the chamber, and wherein the third portion is configured to provide access via an instrument to a portion of the biological sample.

43. The device of claim 38, wherein the chamber further comprises an optical lens coupled to the first portion of the interior wall, wherein the optical lens is configured to orient the light from the optical source and towards the biological sample.

44. The device of claim 38, wherein the chamber further comprises an optical lens coupled to the second portion of the interior wall, wherein, if an additional light is emitted from the biological sample when the biological sample is exposed to the light, then the optical lens is configured to orient the additional light from the biological sample and towards the detector.

45. The device of claim 38, wherein the light is a laser light.

46. The device of claim 38, wherein the light is a light sheet.

47. The device of claim 38, wherein the detector is for selective plane illumination microscopy.

48. The device of claim 38, wherein the detector is a microscope.

49. The device of claim 38, wherein the chamber is configured to hold a liquid.

50. The device of claim 38, wherein the container is a capillary tube.

51. A system comprising:
a container configured to hold a biological sample; and
a device operatively coupled to the container, wherein the device comprises:
(a) a chamber configured to receive the container when the container is holding the biological sample, wherein the chamber comprises:
(i) a first end configured to receive at least a portion of the container;
(ii) a second end; and
(iii) an interior wall connecting the first end and the second end, wherein the interior wall comprises:
(1) a first portion disposed between the first end and the second end, wherein the first portion is configured to permit directing of a light towards the biological sample, wherein the biological sample is disposed within the chamber; and
(2) a second portion disposed between the first end and the second end, wherein the second portion is configured to permit detection, by a detector, of the biological sample when the biological sample is exposed to the light;
(b) an optical source coupled to the chamber and positioned at the first portion of the interior wall, wherein the optical source is configured to direct the light through the first portion and towards the biological sample; and
(c) the detector, wherein the detector is coupled to the chamber and positioned at the second portion of the interior wall, and wherein the detector is configured to detect the biological sample through the second portion upon exposure to the light.

52. The system of claim 51, wherein the container has an inner diameter between about 0.5 millimeters and about 10 millimeters.

53. The system of claim 51, further comprising a moving unit configured to direct a relative movement between the container and the device, wherein the relative movement comprises insertion of the at least the portion of the container, through the first end of the chamber, and into a space within the interior wall of the chamber.

54. The system of claim 52, wherein the first portion of the interior wall has a first optical axis, wherein the second portion of the interior wall has a second optical axis, and
wherein the moving unit is further configured to extrude the biological sample out of the at least the portion of the container while the biological sample is within the space, such that the chamber holds the extruded biological sample at an intersection of the first optical axis of the first portion and the second optical axis of the second portion.

55. The system of claim 54, wherein the first optical axis of the first portion and the second optical axis of the second portion are substantially perpendicular to each other.

56. The system of claim 51, wherein the first portion of the interior wall and the second portion of the interior wall are disposed at a substantially same distance between the first end and the second end of the chamber.

57. The system of claim 56, wherein the chamber further comprises a third portion, wherein the third portion is disposed at the substantially same distance between the first end and the second end of the chamber, and wherein the third portion is configured to provide access via a tube to at least a portion of the biological sample.
